(19) United States
(12) Patent Application Publication
(10) Pub. No.: US 2009/0054915 A1
(43) Pub. Date: Feb. 26, 2009

(54) OBSTRUCTION OF UTERINE ARTERIES TO TREAT UTERINE FIBROIDS USING MECHANICAL INSTRUMENTS TO TWIST THE VESSELS

(76) Inventors: Peter Meier, Hamburg (DE); Burkhard Peters, Wattenbeck (DE); Axel Hentrich, Hamburg (DE)

Correspondence Address: GREENBERG TRAURIG, LLP, 200 PARK AVE., P.O. BOX 677, FLORHAM PARK, NJ, 07932, US

(21) Appl. No.: 11/843,906

(22) Filed: Aug. 23, 2007

(51) Int. Cl. A61B 17/08; A61F 6/08

(52) U.S. Cl. 606/158; 128/834

(57) ABSTRACT

A device for degenerating a fibroid comprises a gripper mechanism sized and shaped so as to grip a uterine artery and a rotating mechanism attached to the gripper mechanism. The rotating mechanism may be rotated so as to rotate said gripper mechanism, thereby twisting a gripped uterine artery so as to obstruct blood flow through the artery. In a method of degenerating a uterine fibroid, the aforesaid gripping device is used to catch hold of a uterine artery and is rotated in a direction so as to twist the uterine artery about itself, thereby obstructing blood flow to the uterine fibroid. The gripping device is held in place for a period of time needed to degenerate the fibroid.

### FIELD OF THE INVENTION

This invention relates, generally, to the use of mechanical instruments to block the flow of blood through the arteries. More specifically, it relates to the treatment of uterine fibroids by obstruction of the uterine arteries.

### BACKGROUND OF THE INVENTION

Uterine leiomyomas (i.e., fibroids) are extremely common benign tumors, which are located primarily within the uterine muscle (i.e., intramural fibroids), the uterine cavity (i.e., submucosal fibroids) or on the serosal surface of the uterus. Such fibroids occur in approximately 20% to 30% of women older than 30 years of age. Medical treatment is usually sought when the fibroids are associated with menorrhagia, pelvic pain or urinary symptoms, or when they are suspected to be the cause of infertility. Treatment options include medical therapy and various types of surgical intervention.

Hysterectomy is considered to be the definitive surgical treatment for those women who no longer wish to maintain their fertility. Though effective, this method has a number of undesirable characteristics. First is the mortality rate for this procedure, which is approximately 30 times as great as the mortality rate for women who have not had hysterectomies. Further adverse effects of hysterectomies include damage to adjacent organs, including removal of the ovaries, lengthy hospital stays and periods of recovery, and an increased likelihood of cardiac arrest, decreased sexual pleasure, and increases in depression or anxiety. Surgical removal of fibroids without hysterectomy, by any surgical method, presents a risk of recurrence of fibroids or, more often, failure to observe existing fibroids or misidentification of the fibroids that are causing adverse symptoms.

It has been established that fibroids can be treated by non-surgical therapies involving the temporary obstruction of the blood flow within the arteries transporting blood into the uterus. One example of such a treatment is uterine artery embolization (UAE). UAE involves the injection of tiny particles of polyvinyl alcohol (PVA) through blood vessels to block the arteries supplying blood to the fibroids. This blockage of the blood supply causes degeneration of the fibroids leading to their death. However, UAE is performed by radiologists who, typically, are unfamiliar with practices of gynecological care. As of now, UAE's are performed in radiology suites, which have high installation and operational costs and which, therefore, are generally restricted to major medical centers. Also, however UAE is practiced, the movement of the PVA particles is flow-directed and their distribution is not limited to the arteries that supply the fibroids, but may affect blood flow to other areas of the uterine tissue or to the ovaries.

There exists a need for devices and methods that can be used to temporarily obstruct the flow of blood to fibroids. The devices should be relatively inexpensive and simple to apply, and should allow the physician to control the degree by which blood flow is reduced. Various devices and methods for obstructing the uterine arteries have been disclosed in the prior art:

U.S. Pat. No. 6,254,601 discloses methods for penetrating the wall of the vaginal vault near the uterine artery with devices that sense the locations of the anatomical structures and occlude the uterine artery. A number of methods and devices are disclosed. These disclosures are also presented in U.S. Pat. Nos. 6,602,251 and 6,764,488.

U.S. Pat. No. 6,550,482 discloses a clamp for temporarily obstructing the uterine artery. The clamp stretches the wall of the vaginal vault around the artery and applies pressure to stop blood flow.

U.S. Patent Publication No. 2002/0165579 discloses a compression device for distending the wall of the vaginal vault and thus compressing the uterine artery. Doppler ultrasound techniques are used to locate the uterine artery and sense when blood flow has stopped.

U.S. Patent Publication No. 2002/0183771 discloses a compression device that clamps around the uterine artery and the vaginal wall to stop blood flow.

U.S. Patent Publication No. 2002/0188306 discloses a forceps-type clamp that is inserted into the vagina and clamps around the uterine artery and the vaginal wall. Ultrasound sensors are placed on the ends of the clamp to allow location of the uterine artery and sense blood flow. Similar forceps-type clamps are described in a number of other references.

U.S. Patent Publication No. 2002/0124853 is directed to a method of temporarily obstructing blood flow through the uterine artery for a set period of time, then re-establishing blood flow through the artery. A forceps-type clamp is used to compress the artery from both sides.

U.S. Patent Publication No. 2004/0092979 discloses a device with paddles that are used to distend the wall of the vaginal vault around the uterus, thus compressing both uterine arteries at the same time.

U.S. Patent Publication No. 2003/0120286 discloses a clip for encircling and compressing a body lumen, of which a uterine artery is one example.

U.S. Patent Publication No. 2004/0097962 discloses constriction devices that can be deployed to distend the vaginal wall around the uterus and thus obstruct the uterine arteries.

### SUMMARY OF THE INVENTION

The invention, in general, relates to a device and a method for degenerating a fibroid by obstructing the flow of blood through a uterine artery. In general, the device includes a gripper mechanism adapted to grip a uterine artery and a rotating mechanism for rotating the gripper mechanism. In a first embodiment, the gripper mechanism includes a hook and the rotating mechanism includes a shaft continuous with the hook. In a second embodiment, the gripper mechanism includes a pair of prongs within a sheath. The prongs act jointly to push a portion of the wall of the vaginal vault of a female patient around a uterine artery, thereby gripping both the portion off the wall and the artery. When used for degenerating a fibroid, each of the embodiments twists the uterine artery about itself so as to obstruct blood flow through the fibroid for a time sufficient to degrade the fibroid.

It should be understood that the embodiments described above are merely exemplary and that additional embodiments may be realized that are within the scope of the invention. The invention is further described in the Detailed Description of the invention presented below.

### BRIEF DESCRIPTION OF THE DRAWINGS

For a more complete understanding of the present invention, reference is made to the following detailed description of the present invention considered in conjunction with the accompanying drawings, in which:

FIG. 1 is a perspective illustration of a hook-shaped device for twisting a uterine artery according to an embodiment of the invention.

FIG. 2 is an illustration of the device of FIG. 1 positioned to capture a uterine artery.

FIG. 3 illustrates the device of FIG. 1 in position after having twisted a uterine artery, the device being shown in combination with an optional pessary.

FIG. 4 illustrates a two-pronged device in an extended position for twisting a uterine artery according to another embodiment of the invention.

FIG. 5 illustrates the device of FIG. 4 in a retracted position, a portion being broken away to facilitate consideration and discussion.

FIG. 6 illustrates the device of FIG. 4 in an extended position to capture a uterine artery.

FIG. 7 illustrates the device of FIG. 4 in a retracted position after capturing a uterine artery.

FIG. 8 illustrates the device of FIG. 4 in position after having twisted a uterine artery, the device being shown in combination with an optional pessary.

### DETAILED DESCRIPTION OF THE INVENTION

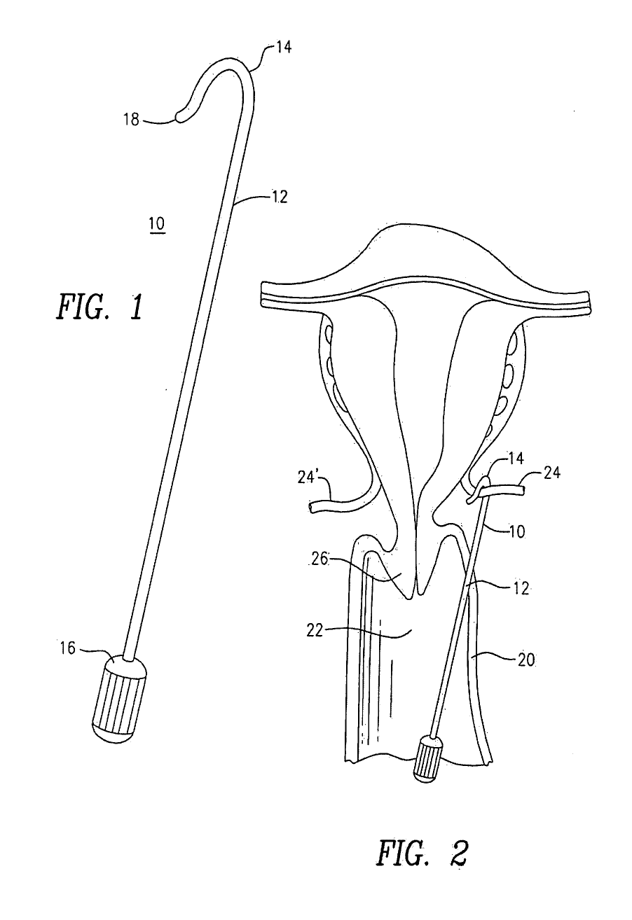

With reference to FIG. 1, a hook-shaped device 10 for catching hold and twisting a uterine artery comprises a shaft 12 having a hook 14 at one end of the shaft 12 and a handle 16 at the other end. The tip 18 of the hook 14 is bent away from the body of the hook 14, reducing the likelihood that the tip 18 will become snagged on any tissue ensnared by the hook 14. The shaft 12, hook 14, handle 16 and tip 18 may be manufactured as a single piece. The shaft 12 and/or handle 16 may be roughened to increase friction at the surface of the device 10 and make it easier to grip. The device 10 may be made of a biologically inert metal, a rigid plastic, or other rigid material. If any portion of the device 10 is to remain inside the body after use, it may be made of a biodegradable polymer. The bioabsorbable polymers that can be used to make devices according to the present invention include conventional biocompatible, bioabsorbable polymers including polymers selected from the group consisting of aliphatic polyesters, poly(amino acids), copoly(ether-esters), polyalkylene oxalates, polyalkylene diglycolates, polyamides, tyrosine derived polycarbonates, poly(iminocarbonates), polyorthoesters, polyoxaesters, polyamidoesters, polyoxaesters containing amine groups, poly(anhydrides), polyphosphazenes, poly(propylene fumarates),. absorbable poly(ester urethanes), biomolecules (i.e., biopolymers such as collagen, elastin, bioabsorbable starches,. etc.) and combinations and blends thereof. The polyoxaesters include the polymers based on 3,6-dioxaoctanedioic acid, 3,6,9-trioxaundecanedioic acid, and the diacid known as polyglycol diacid, which can be made from the oxidation of low molecular weight polyethylene glycol. Currently, aliphatic polyesters are among the preferred absorbable polymers for use in ranking the implants according to the present invention. Aliphatic polyesters can be homopolymers, copolymers (random, block, segmented, tapered blocks, graft, triblock, etc.) having a linear, branched or star structure. Suitable monomers for making aliphatic homopolymers and copolymers may be selected from the group consisting of, but are not limited, to lactic acid (both L- and D-isomers), lactide (including L-, D-, and meso-lactide), glycolic acid, glycolide, ε-caprolactone, p-dioxanone (1,4-dioxan-2-one), trimethylene carbonate (1,3-dioxan-2-one), and combinations thereof. Aliphatic polyesters can be hornopolymers, copolymers (random, block, segmented, tapered blocks, graft, triblock, etc.) having a linear, branched or star structure; alternately they can be a component of a cross-linked network. It is to be understood that exemplary bioabsorbable, biocompatible polymers may be generally synthesized by a ring-opening polymerization of the corresponding lactone monomers or by polycondensation of the corresponding hydroxy-acids, or by combinations of these two polymerization methodologies. Thus as used herein, the term “polyglycolide” is understood to include polyglycolic acid. Further, the term “polylactide” is understood to include polymers of L-lactide, D-lactide, meso-lactide, blends thereof, and lactic acid polymers had copolymers in which other moieties are present in amounts less than 50 mole percent. Other aliphatic polyesters that may provide utility include the hydroxybutyrates and polyhydroxyvalerates.

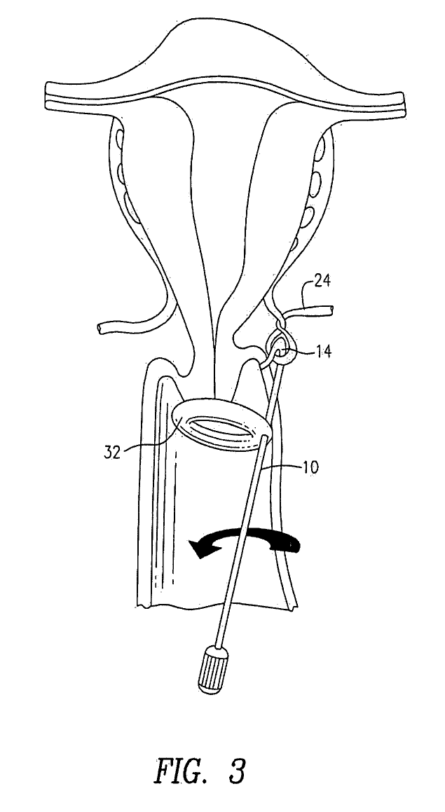

Application of the device 10 is illustrated in FIGS. 2 and 3. Referring to FIG. 2, the portions of the female anatomy pertinent to the application of the hook-shaped device 10, and of devices discussed hereinafter, include the wall 20 of the-vaginal vault 22, the uterine arteries 24, 24′ and the cervix 26.

First, an incision (not shown) is made in the wall 20 of the vaginal vault 22 to expose the uterine artery 24 and the artery 24 is dissected. The hook 14 is inserted through the incision and positioned over the uterine artery 24 so as to ensnare it. The hook 14 may be put into position using a forceps (not shown) or other device capable of releasably gripping the device 10.

Turning to FIG. 3, in a preferred embodiment, the shaft 12 passes through the body of an optional pessary 32 which surrounds the cervix 26. The pessary 32 allows the shaft 12 to be moved along the longitudinal axis of the shaft 12, but may provide some resistance to its rotational movement. The hook-shaped device 10 may be provided separately from the pessary 32, and inserted therethrough, or they may be provided together in a single device. The hook-shaped device 10 is then rotated so that the hook 14 twists the uterine artery 24 about itself so as to completely block blood flow. The shaft 12 is prevented from rotating back towards its original position through a friction fit within the body of the pessary 32, or by a catch (not shown) inside of the pessary 32. Suitable catches include one or two-sided clamps, clips or other grasping devices.

The hook-shaped device 10 is held in position for the length of time needed to degenerate or kill the fibroid without killing adjacent tissues. Preferably, blood flow through the uterine artery 24 is blocked for about 6 to 8 hours, after which time the device 10 is counter-rotated so as to untwist the artery, allowing blood flow to resume. The uterine artery 24 can then be freed from the hook 14 by lifting the hook 14 away from the artery. The device 10 may be shaken gently, if necessary, to free the uterine artery 24 from the hook 14. When the uterine artery 24 has been freed from the hook 14, the hook 14 may be retracted through the incision and removed from the vaginal vault 22.

During the procedure, it is important that the position of the hook-shaped device 10 is known relative to the uterine artery 24, so that the device is not mistakenly applied to another blood vessel or to the ureter (not shown). The position of the hook-shaped device 10 relative to the uterine artery 24 may be determined by any of a number of imaging techniques and/or techniques for monitoring the flow of blood through blood vessels.

Appropriate sensors for imaging and/or blood flow detection include blood flow sensors, sound sensors, pressure sensors, or electromagnetic radiation sensors (e.g., X-ray detectors). Sensors may be mounted on the hook-shaped device 10, on the forceps or other tool used to place or remove the hook-shaped device 10, or on implements temporarily attached to the hook-shaped device during insertion. Since any sensor that is used will have associated wiring it is preferable to use an implement that can be removed after the hook-shaped device 10 is applied to the uterine artery 24. In the absence of such an implement, the patient may be able to move about comfortably with the hook-shaped device 10 in place.

Techniques that may be used include direct visual examination, abdominal ultrasound, Doppler ultrasound, X-ray detection, sound detection, and angiography. Direct visual examination is the preferred method. Ultrasound techniques are also of value because they are reliable, real-time techniques for imaging the position of the hook-shaped device 10 in relation to the uterine artery 24 while the procedure is underway. Doppler ultrasound techniques provide the additional ability to determine when blood flow ceases or is restored. Optical fibers may also be used to illuminate the organs, and transmit images to an optical viewer. Adaptations of suitable techniques for use with the hook-shaped device 10, or other devices that may be discussed herein, will be apparent to a person skilled in the application of such techniques to surgical procedures.

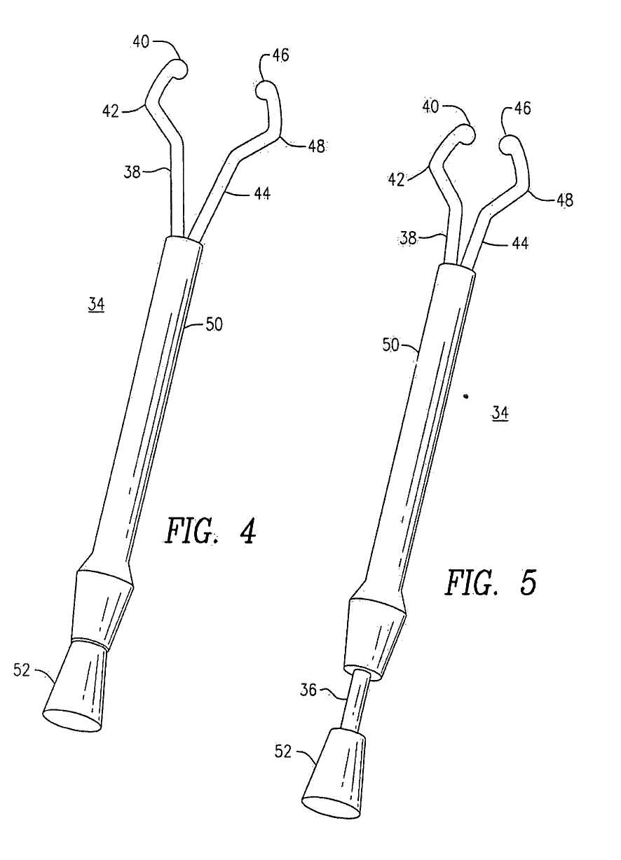

Turning to FIGS. 4 and 5, a two-pronged device 34 comprises a shaft 36 (see FIG. 5), which is bifurcated into a prong 38, having a free end 40 and a bent portion 42 adjacent the free end 40, and a prong 44, also having a free end 46 and a bent portion 48 adjacent the free end 46. Each of the bent portions 42, 48 has a concave (i.e., open) side, the concave sides being oriented such that they face each other. The shaft 36 and prongs 38, 44 may be formed as a single piece or as two or more pieces secured to each other. The free ends 40, 46 are blunted to minimize the chance of snagging or puncturing the tissues that they may contact. The shaft 36 is encompassed by a sheath 50, as are portions of the prongs 38, 44. The outer surface of the sheath 50 may be roughened to increase friction at the surface of the sheath 50, and make it easier to grip. The interior of the sheath 50 is shaped such that the sheath 50 may move forward (i.e., toward the prongs 38, 44) or backward (i.e., away from the prongs 38, 44) along the shaft 36 and the prongs 38, 44, but the shaft 36 and the prongs 38, 44 may not rotate within the sheath 50. A handle 52 may be provided at the end of the shaft 36 opposite the prongs 38, 44 such that the shaft 36 and the prongs 38, 44 may be held in place while the sheath 50 is moved. The prongs 38, 44 are resilient and are flared such that the free ends 40, 46 move toward from each other as the sheath 50 is moved forward from its position in FIG. 4 to its position in FIG. 5.

As with the hook-shaped device 10, the various components of the two-pronged device 34 may be may be made of a biologically inert metal, a rigid plastic, or other rigid material. The two-pronged device 34 is not intended to penetrate, the wall 20 of the vaginal vault 22, so it is less likely that the two-pronged device 34 would be left in place after use. Therefore, there would be less advantage in making components of the two-pronged device 34 out of biodegradable polymers.

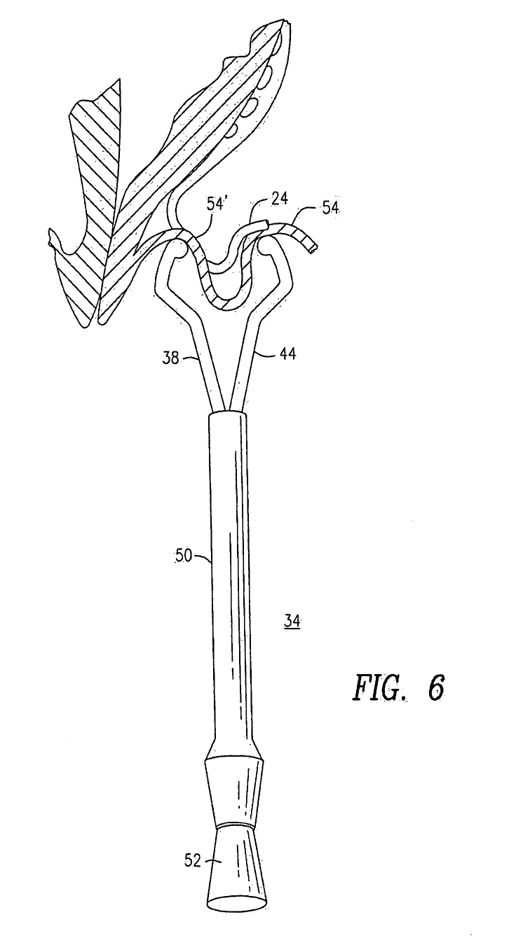

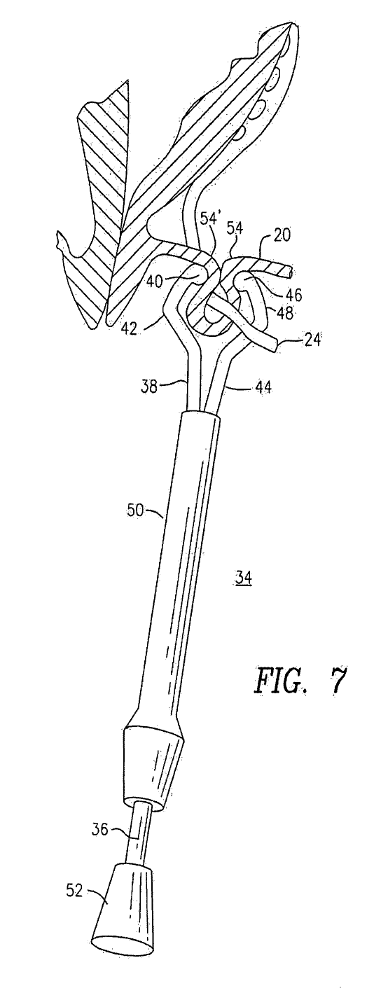

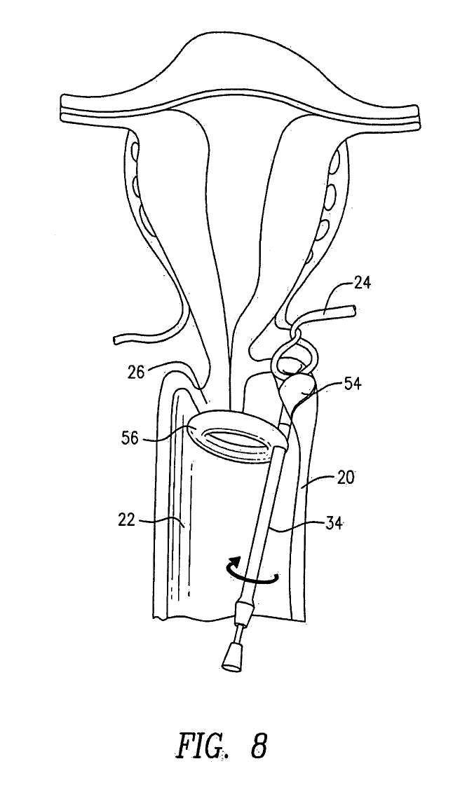

A preferred application of the two-pronged device 34 to the obstruction of the uterine artery 24 is illustrated in FIGS. 6 through 8. The method used is non-invasive and, thus, does not require an incision through the wall 20 of the vaginal vault 22.

Referring to FIGS. 6 and 7, the device 34 is advanced with the prongs 38, 44 extended toward the vaginal vault 22 in an area near the uterine artery 24. The prongs 38, 44 press against the wall of the vault 22, stretching portions 54, 54′ of the wall so that they extend past the uterine artery 24 on both sides. When the bent portions 42, 48 are positioned adjacent the uterine artery 24, the sheath 50 is moved forward so that the free ends 40, 46 press the stretched portions 54, 54′ of the wall 20 of the vaginal vault 22 against each other, in such a manner that they entwine the uterine artery 124 (see FIG. 7). As can be seen in FIG. 7, the bent portions 42, 48 of the prongs 38, 44 are sized and shaped to surround a portion of the wall 20 of the vaginal vault 22 while it is wrapped around the uterine artery 24.

Referring to FIG. 8, the entire device 34 is then rotated so that it twists the wall 20 of the vaginal vault 22 and the uterine artery 24 such that the blood flow through the artery is completely blocked. The prongs 38, 44 are prevented from rotating back to their original position by the internal structure of the sheath 50, which itself is held in position through a friction fit within an optional pessary 56 that surrounds the cervix 26, or by a catch (not shown) inside of the pessary 56. The two-pronged device 34 may be provided separately from the pessary 56 and inserted therethrough, or they may be provided together as a single device. Suitable catches include one or two-sided clamps, clips or other grasping devices.

As discussed above with regard to the hook-shaped device 10, the two-pronged device 34 is held in position for the length of time needed to degenerate or kill the fibroid, after which the two-pronged device 34 is rotated so as to untwist the wall 20 of the vaginal vault 22, and thus untwist the uterine artery 24. The sheath 50 is then moved backward to enable the free ends 40, 46 to move away from each other. The two-pronged device 34 may be shaken gently to release the free ends 40, 46 of the prongs 38, 44 from the vaginal wall 20, if necessary. Means for determining the position of the two-pronged device 34 or the flow of blood through the uterine artery 24 would be the same as those discussed with regard to the hook-shaped device 10, discussed above.

It should be understood that the embodiments described herein are merely exemplary and that a person skilled in the art may make many variations and modifications thereto without departing from the spirit and scope of the present invention. For example, the method of the present invention is meant to include the use of any device that is capable of catching and twisting a blood vessel to reduce the flow of blood therethrough. All such variations and modifications, including those discussed above, are intended to be included within the scope of the invention, which is described, in part, in the claims presented below.

We claim:

1. A device for degenerating a fibroid, comprising a gripper mechanism sized and shaped so as to grip a uterine artery and a rotating mechanism attached to said gripper mechanism, said rotating mechanism capable of being rotated so as to rotate said gripper mechanism and thereby twist a gripped uterine artery in such a manner that blood flow therethrough is substantially obstructed.

2. The device of claim 1, wherein said gripper mechanism includes a hook and said rotating mechanism includes a shaft that is attached to said hook.

3. The device of claim 2 further comprising a pessary having a toroidal body, a portion of said shaft passing through said body of said pessary such that said shaft is movable relative to said pessary in a direction parallel to a longitudinal axis of said shaft.

4. The device of claim 1, wherein said gripper mechanism includes a first prong and a second prong, and said rotating mechanism includes a shaft that is continuous with said prongs.

5. The device of claim 4, further comprising a sheath surrounding a portion of said shaft and a portion of said prongs such that it is movable toward and away from said prongs.

6. The device of claim 5, wherein each of said prongs has a free end remote from said sheath, said free ends being movable toward each other in response to the movement of said sheath toward said prongs and being movable away from each other in response to the movement of said sheath away from said prongs.

7. The device of claim 6, wherein each of said prongs has a bent portion proximal to its free end that is shaped so as to have a concave side, said concave side of said first bent portion facing said convex side of said second bent portion, and both bent portions being sized and shaped to as to receive a uterine artery between them.

8. The device of claim 7, further comprising a pessary having a toroidal body, a portion of said sheath passing through said body of said pessary such that said pessary allows said sheath to be moved along a longitudinal axis of said sheath.

9. A method of degenerating a fibroid, said method comprising the steps of:
gripping a uterine artery with a gripper mechanism; and
rotating the gripping mechanism so as to twist the uterine artery about itself until blood flow to the fibroid is substantially obstructed.

10. The method of claim 9, further comprising the step of holding the gripper mechanism in position so as to maintain obstruction of the blood flow for a time sufficient to degrade the fibroid.

11. The method of claim 10, further comprising the steps of:
counter-rotating the gripper mechanism so as to untwist the blood vessel, thereby restoring blood flow through the uterine artery; and
freeing the uterine artery from the gripper mechanism.

12. The method of claim 11, wherein said rotating step includes the step of entwining the uterine artery with an adjacent portion of a patient's vaginal wall.
